(19) United States
(12) Patent Application Publication
(10) Pub. No.: US 2019/0358106 A1
(43) Pub. Date: Nov. 28, 2019

(54) METHOD AND APPARATUS FOR IMPROVED PHYSICAL REHABILITATION OF PATIENTS WITH IMPAIRED MOBILITY

(71) Applicant: Cynthia Barney, Callahan, FL (US)

(72) Inventor: Cynthia Barney, Callahan, FL (US)

(21) Appl. No.: 16/422,542

(22) Filed: May 24, 2019

Related U.S. Application Data
(60) Provisional application No. 62/675,830, filed on May 24, 2018.
(60) Provisional application No. 62/675,831, filed on May 24, 2018.

(51) Int. Cl. A61G 7/057; A61F 7/08; A61N 1/04

(52) U.S. Cl. CPC A61G 7/05707; A61G 7/05723; A61G 7/05769; A61F 2007/0071; A61N 1/0404; A61G 2203/46; A61G 2203/34; A61F 7/08

(57) ABSTRACT

The present disclosure provides apparatus and method for improving rehabilitation of patients with limited mobility. The apparatus comprises a foam block, with a plurality of foam support pylons thereon, creating a nearly planar top cut according to the support pylons. The foam support pylons have the desirable effect of mitigating pressure and blood pooling at undesirable sites on the patient's body. The method comprises creating such a foam block and placing it under the patient's body in the patient's bed. The support pylons may have sensors or be capable of receiving changes to their physical structure to further facilitate rehabilitation. This can be achieved using electrical wires running through trenches between the support pylons.

### CROSS REFERENCE TO RELATED APPLICATIONS

The present application claims the benefit of the Provisional Patent Application Ser. No. 62/675,830, filed May 24, 2018 and to Provisional Patent Application Ser. No. 62/675,381, filed May 24, 2018; the contents of each of which are relied upon and incorporated herein by reference.

### FIELD OF THE DISCLOSURE

The present disclosure relates to a method and apparatus for improved physical rehabilitation of patients with impaired movement abilities.

### BACKGROUND OF THE DISCLOSURE

Substantial medical evidence shows that lying stationary on a bed for prolonged periods of time can cause damaging effects to the body, such as muscular atrophy, blood pooling, and bedsores. However, patients with impaired mobility who are confined to hospital beds often have very little choice. For example, a rehabilitating paraplegic may lie in the same bed for weeks, relying only on attending medical professionals to shift the paraplegic's position every so often.

Such routine shifts are often inadequate. Additionally, patients with impaired mobility often have accompanying nerve damage, and accordingly may not be aware of the patient's own need to shift the patient's weight to avoid further nerve damage or the other deleterious effects of prolonged bedrest.

### SUMMARY OF THE DISCLOSURE

Accordingly, the present invention provides a method and apparatus for improving the physical rehabilitation of patients with impaired mobility. According to the present disclosure, multiple support pylons are coupled to a base. Each pylon may be independently compressed. In addition, in some embodiments, a length of one or more individual pylons may be altered to better conform to a shape of a body portion of a patient, based upon a support need and a site of sensitive tissue. The structure of these pylons allows for easy patient movement, and in some embodiments, enhanced patient monitoring.

The pylons may be made from a flexible material, such as polyurethane foam or memory foam. The pylons may be comprised of a pylon base and a pylon top. In exemplary embodiments, the pylon base includes a rectangular prism of formable foam or other compressible material. A pylon top may include a larger rectangular prism formed by a pattern of multiple pylons. During use, the base, it is generally situated as a horizontal plane. Pylon tops are arranged along an upper surface of the base and extend upward to support a patient, or a portion of a patient. A pattern, such as a matrix of pylons is included in the pylon top. Other patterns, such as a curved, oval or circular patter are also within the scope of the present invention.

In some embodiments, a top surface of one or more pylons may be essentially planar. In other embodiments, a top surface of one or more pylons may include an arcuate contour or a pointed contour.

In another aspect, a base may have circular holes cut on the sides, creating trenches between pylon bases. The trenches may be used to route tubes, wires or other conduits to a site within the base and/or pylons. The conduits may traverse the trenches to allow electricity, cooling fluid, or other desirable energetic substances to flow between pylons and into and out of the base.

This allows the pylons to have novel, desirable qualities. For example, in some embodiments, one or more pylon may include a transceiver (such as Bluetooth or WiFi), a Peltier device (to allow generation of electricity), or sensors (to improve patient monitoring). These sensors may be used in conjunction with a generated model of a bed to generate a model of a patient. This, in turn, may allow for better patient monitoring, such as improved detection of potential nerve damage and bedsores. One general aspect includes an apparatus for improved rehabilitation of patients with impaired mobility including a first portion of compressible material forming a support base. The apparatus also may include a second portion of compressible material contiguous with the support base and forming multiple support pylons, each pylon separated from an adjacent pylon by a via. The apparatus also may include a support surface formed by the multiple support pylons. The apparatus may also include multiple trenches, each trench formed below the support surface and between the multiple support pylons.

Implementations may include one or more of the following features. For example, the device may include multiple trenches, each trench formed below the support surface and between the multiple support pylons. The apparatus may also include the apparatus where the first portion of compressible material and the second portion of compressible material include polyurethane foam and at least one support trench includes generally circular channel at a bottom of two adjacent pylons extending a length of the second portion of compressible mate The apparatus may also include examples where the first portion of compressible material and the second portion of compressible material include polyurethane foam and at least one support trench includes a generally circular channel at a bottom of two adjacent pylons extending a length of the second portion of compressible material. The apparatus may also include the example where at least one support pylon further includes a moisture sensor. Examples may include those where the apparatus has at least one support pylon which further includes a moisture sensor. The apparatus may also have at least one support pylon further including a compressive force transducer. The apparatus may also include examples where at least one support pylon further includes channels created within the at least one support pylon to generate airflow.

In some examples, the apparatus may further include a transmitter capable of transmitting sensor information to a smart device using at least one of: wi-fi, bluetooth, or cellular data such as 3G, 4G or 5G.

In some examples, the apparatus may include at least one support pylon which further includes an air pump capable of adjusting a compressible force on at least one support pylon. The apparatus may also include examples where the apparatus further includes wire heating elements running through a trench. The apparatus may also include a thermoelectric converter. The apparatus may also include examples which further include an electrode capable of applying an electrical current to a location on a patient's body.

Methods may include examples where a second portion of compressible material is formed from an imprint of a patient's body. The method may further include placing the rectangular prism-shaped block of compressible material underneath a patient on a patient bed. Methods may include steps as follows, making a circular hole at a bottom portion of the multiple incisions, which circular hole runs from a proximal end of the compressible material to a distal end of the compressible mate. The method may include marking a critical area on location of a patient's body. The method may also include mapping the critical areas to coordinates on the compressible material. The method may also include removing a portion of the compressible material in a pattern based upon the mapping of a critical area to coordinates on the compressible material. The method may also include creating a digital model of the compressible material incorporating the mapped critical areas of the patient's body.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIGS. 1A-1E illustrate several different views of an exemplary embodiment of the apparatus for improving physical rehabilitation of patients with impaired mobility.

FIGS. 1F-1J illustrate several different views of another exemplary embodiment of the apparatus for improving physical rehabilitation of patients with impair mobility.

FIG. 2 illustrates an exemplary embodiment of the method for improving physical rehabilitation of patients with impaired mobility.

### DETAILED DESCRIPTION

The present disclosure provides generally for methods and an apparatus for improving physical rehabilitation of patients with impaired mobility. According to the present disclosure, the apparatus comprises a plurality of support pylons of a size, shape, and compressive (or tensile) strength to support at least a portion of a body weight of a patient. Each pylon may be arranged on a base and have a limited range of free movement along an x-y plane and be one or both of compressible and elongated along a z-axis. This allows one or both of a health care provider and a patient to shift the patient's weight around a supporting surface, such as a hospital bed, chair or gurney with minimal effort in response to feeling too much pressure on a given area of the patient's body. Avoiding a threshold amount of pressure over a prolonged period of time is desirable as it may ameliorate a pressure on a specific area of the patient's body and assist the patient's body in reinitializing a flow of blood into and from an affected area, thus mitigating the risk of blood pooling and bedsores.

Further, in some embodiments, support pylons may be enhanced via the inclusion of sensors, electricity, variable pressure, and computer modeling.

In the following sections, detailed descriptions of examples and methods of the disclosure will be given. The description of both preferred and alternative examples though through are exemplary only, and it is understood that to those skilled in the art that variations, modifications, and alterations may be apparent. It is therefore to be understood that the examples do not limit the broadness of the aspects of the underlying disclosure as defined by the claims.

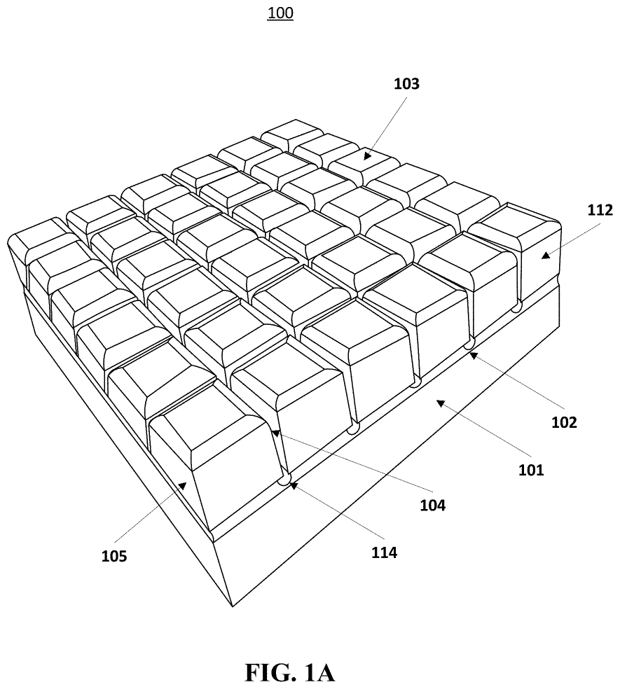

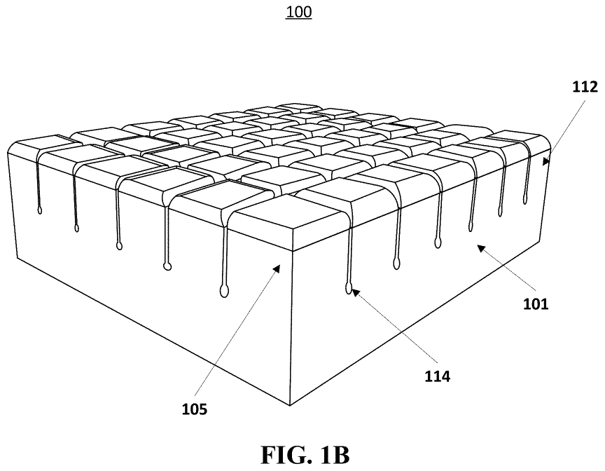

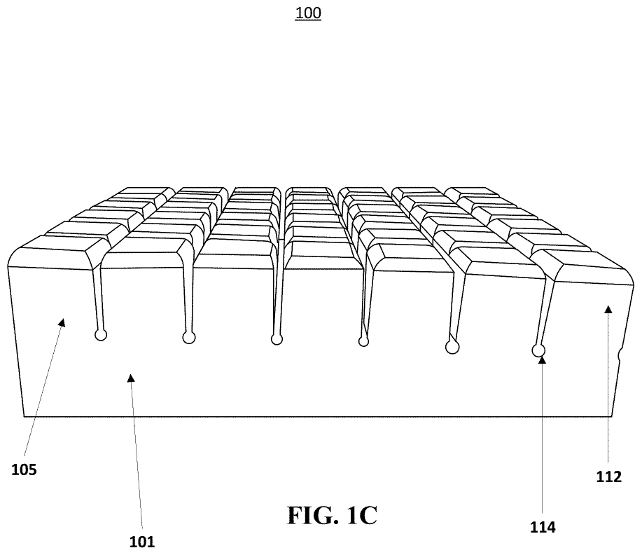

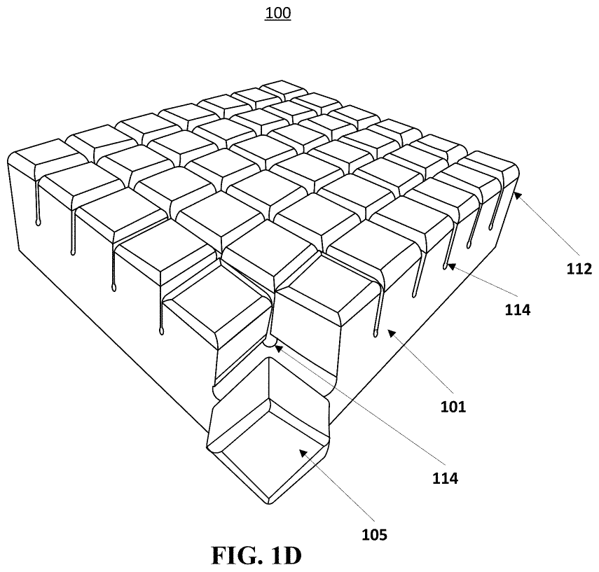

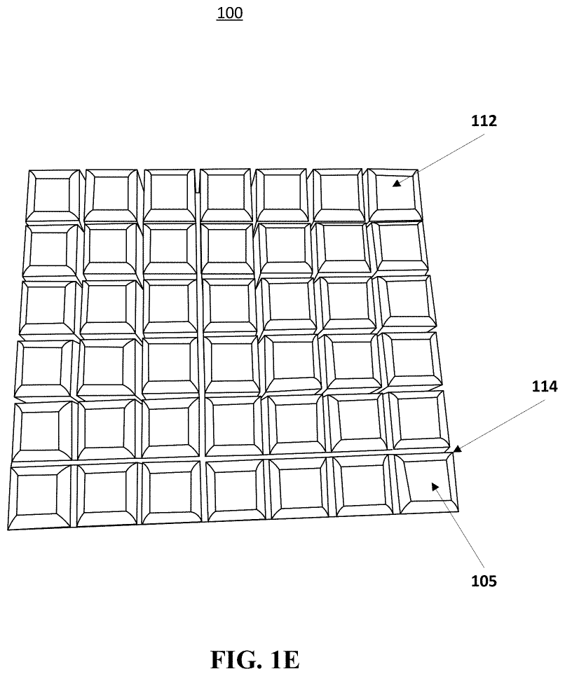

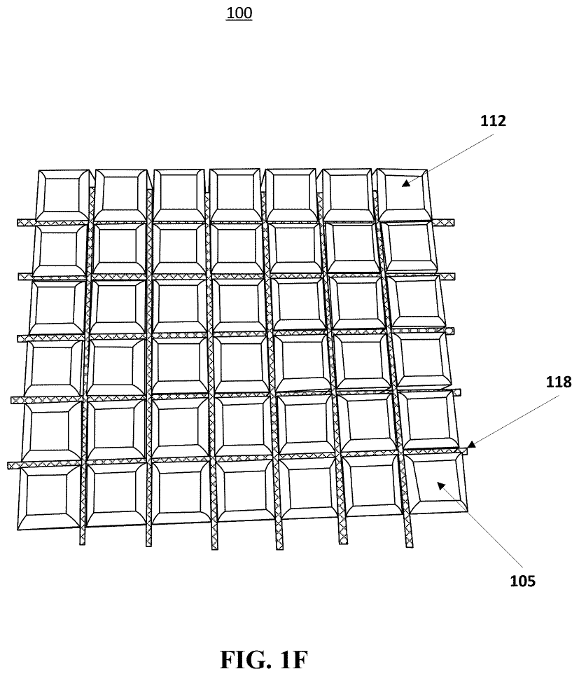

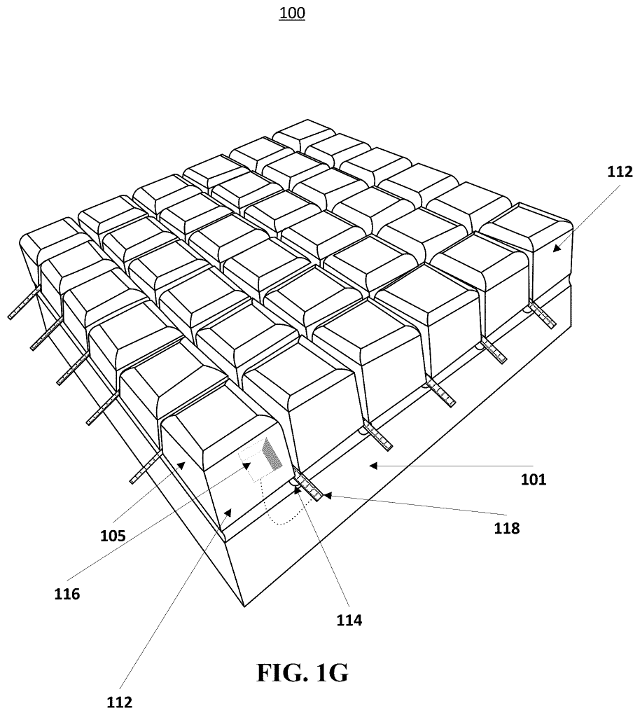

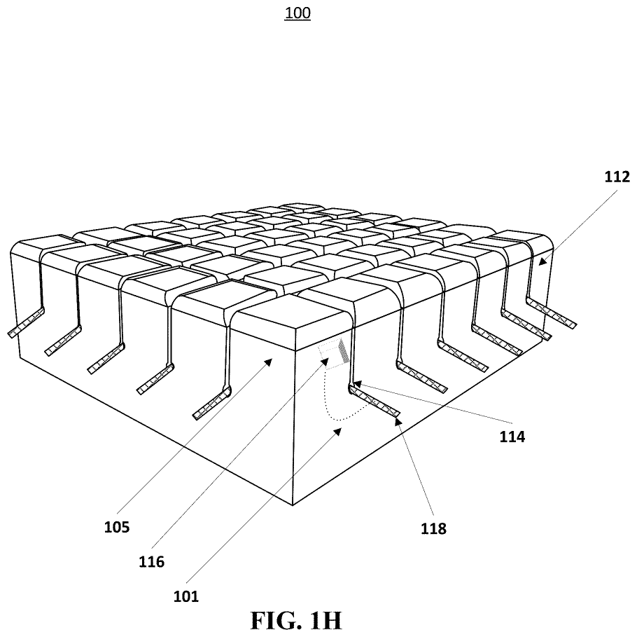

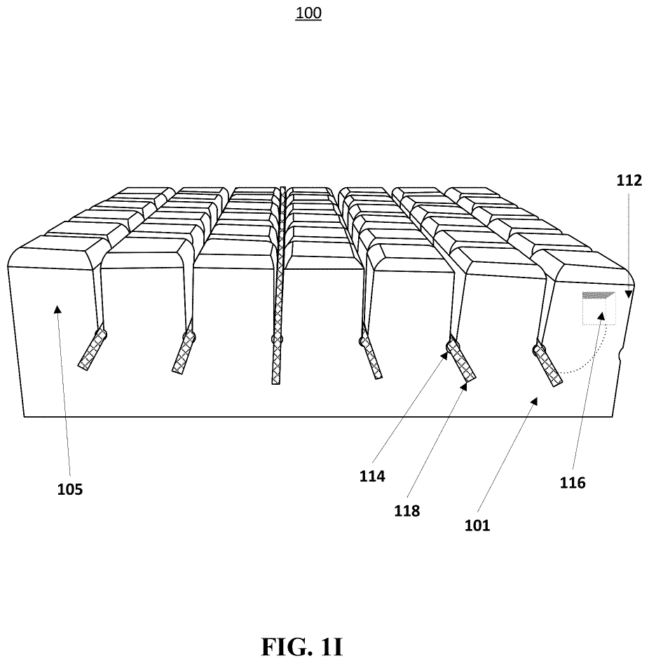

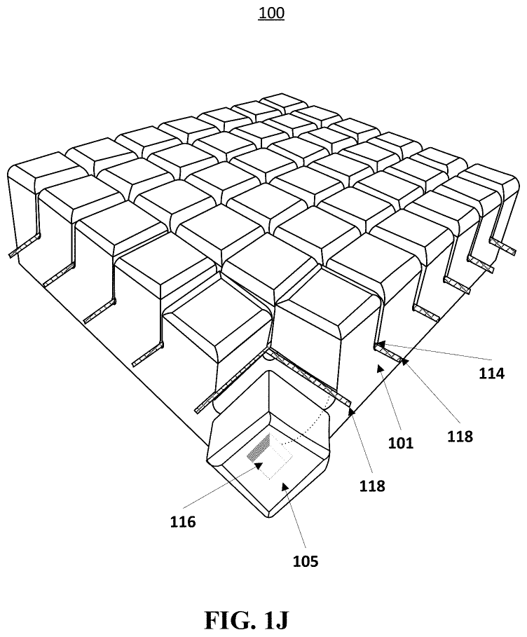

Referring now to FIG. 1A, an exemplary embodiment of an apparatus 100 for improved physical rehabilitation of a patient with impaired mobility, according to some embodiments of the present invention is illustrated. As illustrated, the apparatus 100 includes a base 101 with a generally planar top portion 102. Attached to the base are multiple support pylons 105. The support pylons 105 are individually compressible. As illustrated, the support pylons 105 are each generally extend a same distance from the planar top portion 102 of the base 101 to form a relatively planar support surface 103. Other embodiments may include pylons 105 of variant length such the support surface 103 is arcuate or uneven. A via 104 may be formed between two or more individual pylons 105. In some exemplary embodiments, a via 104 may include, for example one to three inch deep channel. Exemplary embodiments include a via 104 at a spacing conducive to provide sufficient support in an adjacent pylon 105 to support a required amount of body weight and also provide a sufficient amount of compression of the adjacent pylon 104 to alleviate detrimental pressure against the patient's body.

In some embodiments, a via 104 width may be widened, such as by removal of material from an adjacent pylon 105 to decrease a compression strength of the adjacent pylon 105 according to a specific need of a patient's condition.

In some exemplary embodiments, a via 104 may be included about every 1.5-2 inches in either planar direction. For example, each via, may be cut into a single piece of compressible material, such as polyurethane foam, memory foam, natural latex foam or other suitable material to form a plurality of support pylons 105. The base 101 can be made from the same material as the plurality of support pylons 105 or a different material. In exemplary embodiments, the base 101 and the plurality of support pylons 105 are made from polyurethane foam, such as memory foam. Memory foam is a desirable material because of its viscosity, density, and softening reaction to body heat, allowing it to quickly mold to a patient's body. A disadvantage of memory foam is its relative flammability, which may be undesirable in applications in which heat flow to one or more of the support pylons 105 is used to assist in blood transport or spot therapy. Accordingly, other embodiments of the present invention may use a less flammable but similarly flexible material. Still other embodiments may use memory foam for a pylon base, with heat flowing through an insulator running therethrough.

Support Pylons

The plurality of support pylons 105 sit on top of the base 101. In some embodiments, the plurality of support pylons 105 are materially contiguous with the base 101. A support pylon 105 may include a foundation (or pylon base 101), a middle structure with a larger cross-sectional area than the foundation, and a top structure which in some embodiments has a smaller cross-sectional area than the middle structure.

In some embodiments, a pylon may include a size, shape and material to provide a threshold level of support and also a threshold amount of compression. For example a foam used to form a pylon 105 may require 35 pounds of pressure to compress the foam 25% of its original height. Other examples may include between about 15 pounds of pressure compress the foam to 25% of its original height and 60 pounds of pressure to compress the foam to 25% of its original height. Preferred foams may include an indentation load deflection of between about 20 ILD to 55 ILD.

In the embodiment shown in FIG. 1, the cross section of three components of the support pylons 105 is approximately rectangular. Because the foundation of each support pylon 105 is smaller than the middle structure, the apparatus 100 may comprise a plurality of trenches 114. The trenches 114 and the smaller foundation of the support pylons 105 give each support pylon 105 and some freedom of movement within an associated x-y plane. Additionally, because each support pylon 105 may be made from a flexible material, each support pylon 105 may be compressible, giving each support pylon 105 an additional degree of freedom of movement along the z-axis. This compressibility trait also applies along the x- and y-axes. FIGS. 1B-1E show other embodiments of the present invention. FIG. 1D, for example, illustrates the aforementioned compressibility trait of support pylon 105.

### Support Pylons May have Sensors and Dynamic Structural Properties to Enhance Patient Rehabilitation

In some embodiments, such as those shown in FIG. 1G at least one support pylon includes a sensor 116. The sensor may include one or more of: pressure sensor, compressive force sensor, tensile force sensor, heat sensor, force transducer, moisture sensor, or channels for airflow. In some embodiments, the support pylons are not uniform in one or both of compressive force and tensile strength, so for example, a support pylon typically located near a patient's spine may have more “give” than a support pylon near the patient's shoulder blades. In embodiments in which the pylons can have variable compressive force and variable tensile strength (which may be modulated through, e.g., air pumps that can “inflate” non-porous cavities within the memory foam or laser activation), the physical location of each support pylon may be associated with a model of the bed or the patient's body, allowing for dynamic movement of support pylons with variable tensile strength. For example, in such an embodiment, a support pylon initially located near a patient's spine may have more “give” than a support pylon near the patient's shoulder blades. If the patient shifts to the right by 10 cm, then this is reflected on the model, which may then shift the region of more “give” to the right by 10 cm as well.

In some embodiments, one or more support pylons 105 may be capable of adjusting the tension applied to the part of the patient's body in contact with the support pylon. This may be accomplished by means of, for example, pumping additional air into the constituent foam of the one or more support pylon (or into a constituent, non-porous cavity therein); decreasing the tension in a spring within the one or more support pylon; or a piezoelectric device adjustable by means of a handheld smart device.

Some of the foregoing uses may be facilitated by electrical energy. Such uses may include, for example, pumping the foam to increase density and transmitting sensor readings wirelessly. This electrical energy may be provided in several ways. Running electrical wire through the trenches is possible, as shown in wire 118 in FIGS. 1F-1J, but not necessarily optimal. FIG. 1G, for example, shows sensor 116 energized by connection to wire 118. In some embodiments, the electrical energy may be produced by means of a thermoelectric converter, such as a Peltier device. This may have the desirable effect of cooling support pylons, such as pylons receiving too much heat or recently vacated pylons with residual body heat, as well as powering any sensors or devices requiring electricity. Thus, the characteristic temperature gradient required for operation of a Peltier thermoelectric generator may be created between the patient's body and a bed or other similar apparatus. In some embodiments, a support pylon 105 or the base 101 may contain a thermoelectric battery to store residual energy.

In some embodiments, the top of one or more support pylons may comprise an electrode or piezoelectric device able to apply current to a desirable part of the body. The electrode or piezoelectric device may be connected to an electric current source exterior to the base, or may be generated as described above using a Peltier device.

In other embodiments, it may not be possible to power data sensors through internal power sources. In these circumstances, a patch similar to the patch used in electrocardiogram treatments may be used to take measurements of heat and pressure on parts of the body. The tension in affected support pylons may be adjusted accordingly.

Support Pylons can Provide Caregivers with New, Highly Accurate Information

In some embodiments, either the base or at least one support pylon can further comprise a transmitter or transducer (illustrated as sensor 116) capable of transmitting readings from sensors on any support pylon. The transmitter can transmit information wirelessly such as, for example, by Bluetooth, Wi-Fi, or cellular data (e.g., 3G, 4G). Caregivers may access this information via, for example, wireless receiver, such as a smart device. This is highly desirable because it provides caregivers with necessary information, such as whether a certain pylon is under too much pressure (perhaps indicating that the patient's position needs to be changed) or whether a pylon receives an acute spike in thermal energy (perhaps indicating a spill of a hot liquid, or the presence of a medical condition diagnosable by such an acute thermal energy reading, such atherosclerosis).

### The Support Pylons can Facilitate Mapping of a Patient's Body

In some embodiments, the apparatus 100 may be mapped from an imprint of the individual patient's body. This allows for specific tailoring of the support pylons 105 to the patient's needs or pressure points. For example, while a generic apparatus is contemplated with generic assumptions for the location of the patient's spine, shoulder blades, etc., more effective is an apparatus built to support the locations of the patient's actual spine, shoulder blades, etc. Moreover, in embodiments in which the tension of each support pylon may be adjusted, the preliminary location of body parts may be shifted according to the patient's movement. For example, the initial mapping of the patient's body may show that patient's spine (modeled as a one-dimensional structure) will initially rest in an x-y plane in the range (x₀,y₀) to (x₀,y₁). If the patient moves to the patient's left by 10 cm, then the mapping will update to accommodate the spine resting in the range (x₀-10,y₀) to (x₀-10, y₁).

### A Method for Improving Rehabilitation Using the Support Pylons

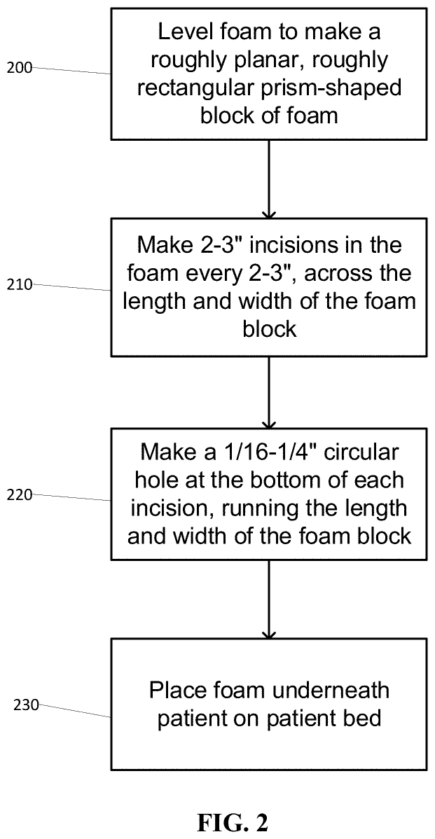

FIG. 2 discloses a method for improving physical rehabilitation of patients with limited mobility. At 200, a piece of foam is leveled. As described above, in some embodiments the foam comprises memory foam. The foam should be leveled so as to create a roughly rectangular prism-shaped block of foam. At 210, 2-3″ incisions are made every 2-3″ along the foam block. This turns the block of foam into a rectangular prism-shaped base, on top of which sits a plurality of support pylons.

At 220, small circular incisions are cut across the length and width of each block. This is desirable because the resulting trench between support pylons allows greater flexibility of movement of each pylon. The 220 step creates a long, cylindrical hole at the bottom of each incision. Finally, at 230, the entire foam shape—base, support pylons, and intervening trenches—is placed underneath a patient on the patient's bed, chair or gurney. This improves the rehabilitation process for patients with limited mobility, or patients who will stay in bed for prolonged periods of time, because it allows for easy manipulation of the pressure exerted on the bed. Accordingly, no one area on the patient's body is subjected to an undue amount of pressure for prolonged periods of time, and blood is less likely to pool in any one area.

### CONCLUSION

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. Nevertheless, it will be understood that various modifications may be made without departing from the spirit and scope of the claimed disclosure.

What is claimed is:

1. An apparatus for improved rehabilitation of patients with impaired mobility, which apparatus comprises:
a first portion of compressible material forming a support base;
a second portion of compressible material contiguous with the support base and forming multiple support pylons, each pylon separated from an adjacent pylon by a via;
a support surface formed by the multiple support pylons, the support surface; and
multiple trenches, each trench formed below the support surface and between the multiple support pylons.

2. The apparatus of claim 1, wherein the first portion of compressible material and the second portion of compressible material comprise polyurethane foam and at least one support trench comprises generally circular channel at a bottom of two adjacent pylons extending a length of the second portion of compressible material.

3. The apparatus of claim 2, wherein at least one support pylon further comprises a moisture sensor.

4. The apparatus of claim 2, wherein at least one support pylon further comprises a heat sensor.

5. The apparatus of claim 2, wherein at least one support pylon further comprises a compressive force transducer.

6. The apparatus of claim 2, wherein at least one support pylon further comprises channels created within the at least one support pylon to generate airflow.

7. The apparatus of claim 2, wherein at least one support pylon further comprises a pressure sensor.

8. The apparatus of claim 2, wherein the apparatus further comprises a transmitter capable of transmitting sensor information to a smart device using at least one of: Wi-Fi, Bluetooth, or cellular data such as 3G or 4G.

9. The apparatus of claim 2, wherein at least one support pylon further comprises an air pump capable of adjusting a compressible force on at least one support pylon.

10. The apparatus of claim 2, wherein at least one support pylon further comprises at least one spring capable of adjusting a compressible force on at least one support pylon.

11. The apparatus of claim 2, wherein the apparatus further comprises a wire heating elements running through a trench.

12. The apparatus of claim 2, wherein the apparatus further comprises electrical wires running through a trench.

13. The apparatus of claim 2, wherein the apparatus further comprises a thermoelectric converter.

14. The apparatus of claim 2, wherein the apparatus further comprises an electrode capable of applying an electrical current to a location on a patient's body.

15. The apparatus of claim 2, wherein the second portion of compressible material is formed from an imprint of a patient's body.

16. A method for improved rehabilitation of patients with impaired mobility, which method comprises:
leveling a foam quantum to create a roughly rectangular prism-shaped block of compressible material;
forming multiple incisions between two and three inches deep into the compressible material spaced between two and four inches apart across a length and a width of the rectangular prism-shaped block of compressible material; and
placing the rectangular prism-shaped block of compressible material underneath a patient on a patient bed.

17. The method of claim 16, wherein the method further comprises the step of: making a circular hole at a bottom portion of the multiple incisions, which circular hole runs from a proximal end of the compressible material to a distal end of the compressible material.

18. The method of claim 17, wherein the method further comprises the steps of:
marking a critical area on location of a patient's body; and
mapping the critical areas to coordinates on the compressible material.

19. The method of claim 18, wherein the method further comprises the step of:
a. removing a portion of the compressible material in a pattern based upon the mapping of a critical area to coordinates on the compressible material; and
b. creating a digital model of the compressible material incorporating the mapped critical areas of the patient's body.

20. The method of claim 16, wherein the method further comprises the step of: periodically applying heat to specified location of the compressible material.
